(19) United States
(12) Patent Application Publication
(10) Pub. No.: US 2015/0168379 A1
(43) Pub. Date: Jun. 18, 2015

(54) METHOD OF IDENTIFYING AND SCREENING DRUG CANDIDATE FOR PREVENTING AND/OR TREATING ISCHEMIC MYOCARDIAL DISEASE

(71) Applicant: Macau University of Science and Technology, Macau (CN)

(72) Inventors: Pei LUO, Macau (CN); Hua ZHOU, Macau (CN); Geng Ting DONG, Macau (CN)

(73) Assignee: Macau University of Science and Technology, Macau (CN)

(21) Appl. No.: 14/103,851

(22) Filed: Dec. 12, 2013

(51) Int. Cl. G01N 33/50

(52) U.S. Cl. CPC G01N 33/5079

(57) ABSTRACT

The present invention provides a method for identifying a therapeutic drug candidate for preventing and/or treating hypoxia-related heart disease. The present invention also provides a kit for screening a therapeutic drug candidate for preventing and/or treating hypoxia-related heart disease.

### FIELD OF INVENTION

This invention relates to a method for identifying a therapeutic drug candidate for preventing and/or treating hypoxia-related heart disease, in particular, ischemic myocardial disease.

### BACKGROUND OF INVENTION

Ischemic heart disease is a clinical syndrome resulting from myocardial ischemia and characterized by an imbalance between the supply and demand of myocardial blood flow, and myocardial oxygen metabolism. High morbidity and complex pathogenesis of ischemic myocardial disease are highly influential to prognosis. Due to the diversity and changes of myocardial ischemia reperfusion injury, disease/cell models and the evaluating indicators thereof used in the pharmacology study could hardly be confined to a single static pattern. As such, it is important to develop reliable models to study this disease, and thus develop methods for screening and/or diagnosing patients having this disease based on such model so that they can receive appropriate and suitable treatment as soon as possible.

### SUMMARY OF INVENTION

In the light of the foregoing background, it is an object of the present invention to provide a method of identifying a therapeutic drug candidate for preventing and/or treating ischemic myocardial disease, in which such identification is based on a new mechanism of protopanaxatriol-type ginsenosides in preventing and/or treating myocardial ischemia reperfusion injury.

Accordingly, the present invention, in one aspect, provides a method of identifying a therapeutic drug candidate for preventing and/or treating hypoxia-related heart disease comprising:

a) pre-treating myocardial cells with said drug candidate;

b) treating said cells under hypoxia and reoxygenation condition;

c) detecting whether said drug candidate binds with mitofusin-2 in mitochondria; and

d) identifying a drug candidate that performs said binding action of step (c).

In an exemplary embodiment of the present invention, the step (c) further comprises a step of detecting whether said drug candidate inhibits apoptosis of said cells;

wherein a drug candidate is identified as a therapeutic drug for preventing and/or treating said hypoxia-related heart disease if said drug candidate can perform both inhibition of apoptosis and binding action of step (c).

In a further exemplary embodiment of the present invention, the hypoxia-related heart disease is selected from a group consisting of ischemic myocardial disease, myocardial ischemia reperfusion injury and altitude sickness.

In an exemplary embodiment of the present invention, the myocardial cells are treated under hypoxia condition for 1-3 hours.

In an exemplary embodiment of the present invention, the myocardial cells are treated under hypoxia condition for 3 hours.

In a further exemplary embodiment of the present invention, the myocardial cells are treated under reoxygenation condition for 1-4 hours.

In an exemplary embodiment of the present invention, the myocardial cells are treated under reoxygenation condition for 3 hours.

In a further exemplary embodiment of the present invention, the myocardial cells are treated under hypoxia condition for 3 hours and reoxygenation condition for 3 hours.

In yet another aspect, the present invention provides a kit for screening a therapeutic drug candidate for preventing and/or treating hypoxia-related heart disease, comprising myocardial cells pretreated with said drug candidate and treated under hypoxia and reoxygenation conditions; and a protocol for comparing cell viability of said myocardial cells pretreated with said drug candidate and myocardial cells not pre-treated with said drug candidate under hypoxia and reoxygenation conditions, wherein an increase in cell viability of myocardial cells pretreated with said drug candidate over those without pre-treatment is indicative of presence of a prevention/treatment effect of said drug candidate.

In an exemplary embodiment of the present invention, the cells are originated from H9C2 or HL-1 cell line.

In a further exemplary embodiment of the present invention, the hypoxia-related heart disease is selected from a group consisting of ischemic myocardial disease, myocardial ischemia reperfusion injury and altitude sickness.

In a further exemplary embodiment of the present invention, the cells are treated under hypoxia condition for 1-3 hours.

In an exemplary embodiment of the present invention, the cells are treated under hypoxia condition for 3 hours.

In another exemplary embodiment of the present invention, the cells are treated under reoxygenation condition for 1-4 hours.

In another exemplary embodiment of the present invention, the cells are treated under reoxygenation condition for 3 hours.

In a further exemplary embodiment of the present invention, the cells are treated under hypoxia condition for 3 hours and reoxygenation condition for 3 hours.

### BRIEF DESCRIPTION OF FIGURES

FIG. 1 shows cell viability of myocardial cells, i.e. H9C2 cell line, under hypoxia and reoxygenation conditions without any treatment.

FIG. 2 shows cell viability of myocardial cells, i.e. H9C2 cell line, pretreated with different concentrations of Rg1, under hypoxia and reoxygenation for different time periods.

FIG. 3 shows cell viability of myocardial cells, i.e. H9C2 cell line, pretreated with different concentrations of Re, under hypoxia and reoxygenation conditions.

FIG. 4 shows the effect of Rg1 on apoptosis rate of myocardial cells, i.e. H9C2 cell line, under hypoxia and reoxygenation conditions.

FIG. 5 shows cytotoxicity effect of Rg1 on myocardial cells, i.e. H9C2 cell line, under hypoxia and reoxygenation conditions.

FIG. 6 shows the effect of Rg1 on membrane potential of mitochondria of myocardial cells, i.e. H9C2 cell line, going through hypoxia and reoxygenation conditions.

FIG. 7 shows the effect of Rg1 on ROS release in myocardial cells, i.e. H9C2 cell line, under hypoxia and reoxygenation conditions.

FIG. 8 shows the effect of Rg1 on the expression levels of related proteins in mitochondira-reduced apoptosis pathway in myocardial cells, i.e. H9C2 cell line.

FIG. 9 shows the binding of Rg1 with mitofusin-2 in myocardial cells, i.e. H9C2 cell line, under hypoxia and reoxygenation conditions.

### DETAILED DESCRIPTION OF THE PREFERRED EMBODIMENTS

As used herein and in the claims, “comprising” means including the following elements but not excluding others.

In the present invention, inventors studied injury of ischemia reperfusion on heart and regulatory mechanism of ischemic myocardial mitochondria from the perspective of mitochondrial dynamics of living cells. They further investigated how the seven components of protopanaxatriol-type ginsenoside affect dynamic change of mitochondria and exert protection function in the heart in order to demonstrate a novel mechanism for treating ischemic heart disease by ginseng. The two major areas of this study are as follows.

1. Building a Novel Model for Evaluating the Dynamic Change of Mitochondria of Ischemic Myocardial Reperfusion Injury

Hypoxia and reoxygenation conditions for myocardial cells, and technology of using labeled probe of mitochondria of living cells were optimized. Pattern in dynamic change of mitochondria of myocardial cells under hypoxia and reoxygenation conditions was studied by real-time dynamic fluorescent images for single living cells and their mitochondria. The relationship of this pattern with cell morphology and biochemical index was analyzed so as to develop new index for screening and evaluating drugs for treating myocardial ischemia based on dynamic change of mitochondria. The effect of the seven components of protopanaxatriol-type ginsenosides on the survival of myocardial cells and mitochondrial function was evaluated by using, in combination, observation from cell morphology and biochemistry assays.

2. Demonstrating the Mechanism in Treating Myocardial Ischemia Reperfusion Injury using Protopanaxatriol-Type Ginsenosides, with the Dynamic Change of Mitochondria of Protopanaxatriol-Type Ginsenosides Being the Specific Target

The specific regulatory protein which ginseng acts on was identified through mitochondrial division, fusion and varying the level of expression and gene of regulatory protein. Therefore, a novel mechanism by which ginseng acting on dynamic change of mitochondria, regulating mitochondrial function and exerting anti-myocardial ischemia function was illustrated. Efficacy study on the whole animals was conducted on in vitro screening samples so as to create foundation for developing new Chinese traditional drugs of ginseng that specifically act on dynamic change of mitochondria, with heart protection and good in vivo efficacy.

In this invention, in vitro injury induced by hypoxia and reoxygenation conditions in myocardial cell lines (i.e. H9C2 and HL-1) was used as experimental models, which were used to assess the injury of myocardial cells and the protection function of drugs. The experimental models were also used to analyze respiratory function of mitochondria, and changes of division, fusion and movement of mitochondria in myocardial cells induced by hypoxia and reoxygenation. The effects of the seven components of protopanaxatriol-type ginsenosides on dynamic change of mitochondria of ischemic myocardial cells and survival of myocardial cells were studied. A novel mechanism of ginsenosides in regulating function of myocardial mitochondria via dynamic pathway and exerting anti-myocardial ischemia function was studied in details.

Redox-dependent non-fluorescent precursor probe that was able to go through intact cell membrane was used, which can be reduced by active respiratory chain in mitochondria and then fixed at this site. The precursor probe can provide sensitive and clear fluorescent signal, which can be continuously collected by fluorescence microscope system at single living cell level and converted to images that were used to directly analyze dynamic change of mitochondria of myocardial cells before and after hypoxia and reoxygenation injury. Combining classical mitochondrial function and survival index of myocardial cells, the connection between dynamic change and function of myocardial mitochondria during myocardial ischemia reperfusion injury was evaluated. Regulation function of dynamic change of mitochondria during myocardial ischemia reperfusion injury was studied by siRNA interference, mitochondrial division and fusion, and gene expression. The function of dynamic change of mitochondria and in vivo ginsenosides pre-treatment were confirmed using mouse LAD ligation model. The structure-activity relationship of the seven components, Re, Rf, Rg1, 20(S)-Rg2, 20(R)-Rg2, 20(S)-Rh1 and 20(R)-Rh1 from protopanaxatriol-type ginsenosides would illustrate the difference in mechanism of influences of different stereo-isomers of protopanaxatriol-type and combined glycosyl on dynamic change of mitochondria. Characteristic regulatory site would also be located. New ginseng drugs having myocardial protective effect by acting on dynamic change of mitochondria would be developed.

The present invention is further defined by the following examples, which are not intended to limit the present invention. Reasonable variations, such as those understood by reasonable artisans, can be made without departing from the scope of the present invention.

### Example 1

### A Method of Preparing a Model of Myocardial Cell Injury Induced by Hypoxia-Reoxygenation

Myocardial cell lines, H9C2 and HL-1 were respectively seeded into culture dishes. KRB (Krebs-Ringer Bicarbonate buffer) solution replaced normal DMEM medium without the addition of serum. Cells were immediately placed in adjustable hypoxia box (Billups-Rothenberg) connected with gas flowmeter (rate of flow: 25L/min), in which the partial pressure of oxygen in the box was made to decrease from 20 kPa to 0 kPa within one minute. Hypoxia box was placed in a regular incubator for different incubation periods to create hypoxia conditions. Hypoxia box was then opened and the medium was replaced with normal medium containing serum, followed by normal incubation at 37° C. for the time periods corresponding to those in the hypoxia conditions in order to create re-oxygenation conditions.

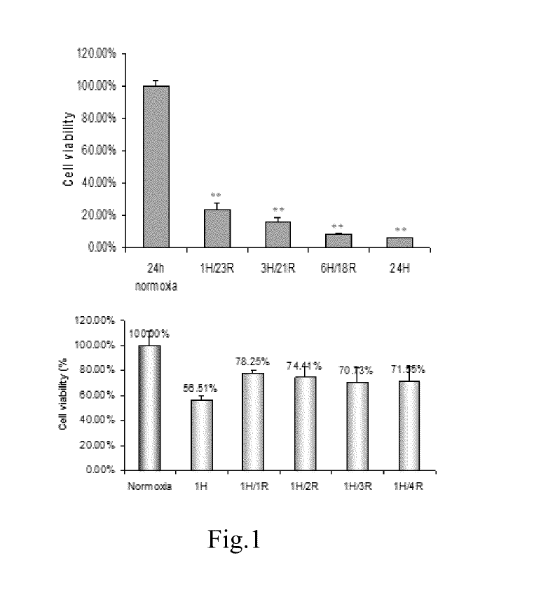

Cell viability was determined under different hypoxia and reoxygenation durations as shown in FIG. 1, in which H denotes hypoxia and R denotes reoxygenation and the number before H/R represents the duration for hypoxia/reoxygenation. For example, “1H/23R” indicates the specific study involves an hour of hypoxia followed by a 23-hour of reoxygenation. It can be seen from FIG. 1 that hypoxia condition could decrease cell viability and subsequent reoxygenation could recover cell viability to a higher level, on comparing with the study in which only hypoxia condition was applied. This result shows the cell model as described above can be used to evaluate the capacity of candidate agents in preventing and/or treating ischemic myocardial disease by determining cell viability of myocardial cells.

### Example 2

### A Method of Pre-treating Myocardial Cells with Components of Protopanaxatriol-Type Ginsenosides

Seven components of protopanaxatriol-type ginsenosides, namely Re, Rf, Rg1, 20(S)-Rg2, 20(R)-Rg2, 20(S)-Rh1 and 20(R)-Rh1, were used to pre-treat myocardial cells. Each component was dissolved with normal medium and DMSO as co-solvent and used to pre-treat cells one hour before hypoxia. Subsequently, medium was replaced with hypoxia medium. The effects of each component on survival, mitochondrial respiration, morphology, and mitochondrial function and movement of myocardial cells were analyzed, and the results thereof were used in candidate agent selection.

### Example 3

### Study on Survival Rate of Myocardial Cells Pre-treated with Components of Protopanaxatriol-type Ginsenosides

Myocardial cells were seeded into 96-well plates and incubated under normal condition for 48 hours, followed by pre-treatment as described by Example 2, and then treated under hypoxia and reoxygenation for different time periods. MTT working solution was added into the cells (10 μl/well) and cells were incubated at 37° C. for 4 hours.

Then 10% SDS buffer solution was added into the cells (100 W/well), avoiding bubbles within the cells. Cells were incubated under 37° C. overnight and then dissolved and mixed on the next day.

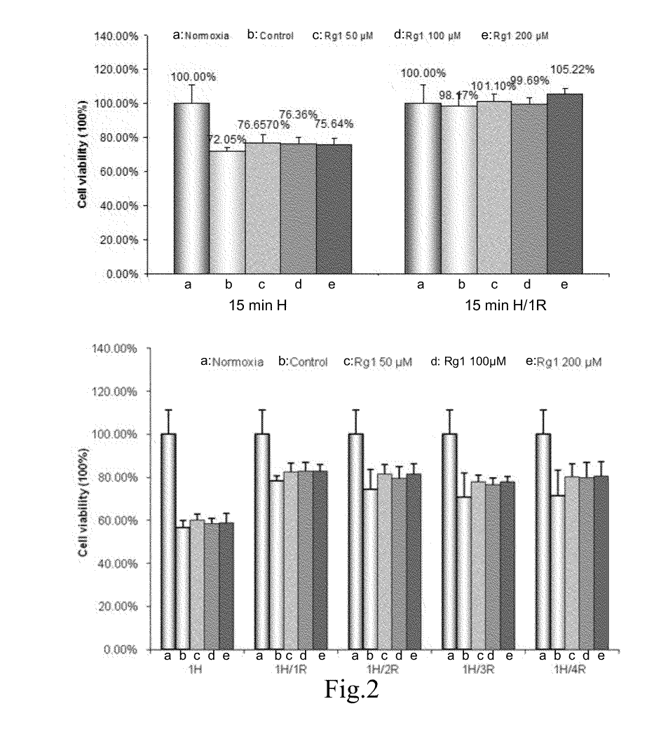

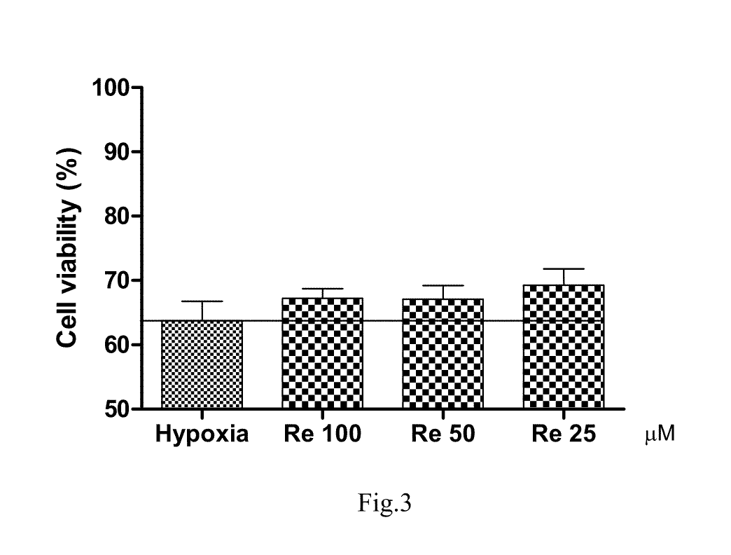

Optical density (OD) of cells was measured at wavelength of 570 nm by multiple-well plate spectrophotometer. Survival rate of the cells incubated under normal condition was set as 100%, and the survival rate of the blank control group was measured simultaneously as baseline for background subtraction. Survival rates of myocardial cells treated under different conditions were calculated by the following formula, and results for pre-treatment with Rg1 and Re are shown in FIGS. 2 and 3 respectively.

Survival rate of cells=(OD of experiment group treated under hypoxia-reoxygenation−OD of blank control group)/(OD of normal control group−OD of blank control group)×100%.

Survival rate of myocardial cells, i.e. H9C2 cell line, pre-treated with Rg1 is shown in FIG. 2. The result shows that Rg1 is capable of increasing survival rate of myocardial cells going through hypoxia and reoxygenation.

Survival rate of myocardial cells, i.e. H9C2 cell line, pre-treated with Re is shown in FIG. 3. The result shows that Re is capable of increasing survival rate of myocardial cells going through hypoxia and reoxygenation.

### Example 4

### Analysis on Membrane Potential of Mitochondria/ROS Release, Apoptosis Rate and Cell Cytotoxicity of Myocardial Cells Using Flow Cytometry

Membrane potential of mitochondria, apoptosis rate, ROS release and cell cytotoxicity of myocardial cells were analyzed by flow cytometry. Upon pre-treatment of cells in different groups with Rg1, Sil (Sildenafil, the positive control drug), DOX (Doxorubicin, a cardiac toxic chemical which can induce H/R damage in myocardial cells) or FCCP (Carbonyl cyanide 4-(trifluoromethoxy) phenylhydrazone), the cells were then treated with hypoxia and reoxygenation conditions, in which the duration of each condition was 3 hours. 1 ml PBS buffer was added into culture dish with mixing. Then Rhodamine 123 (Rh123) was added to a final concentration of 5 μM. Cells were incubated for 10 minutes in dark at 37° C., digested with trypsin, and collected by centrifuge. Supernatant was removed and cells were washed by PBS buffer twice. Fresh PI working solution was added into cells with mixing. Fluorescence intensity of cells were analyzed by flow cytometry (each sample contains more than 10,000 cells). Cell fluorescence image was analyzed with fluorescence intensity of Rh123 as x-axis and fluorescence intensity of PI as y-axis. Three parallel samples were arranged for each treatment group in each study.

### 4.1 Study on Apoptosis Rate

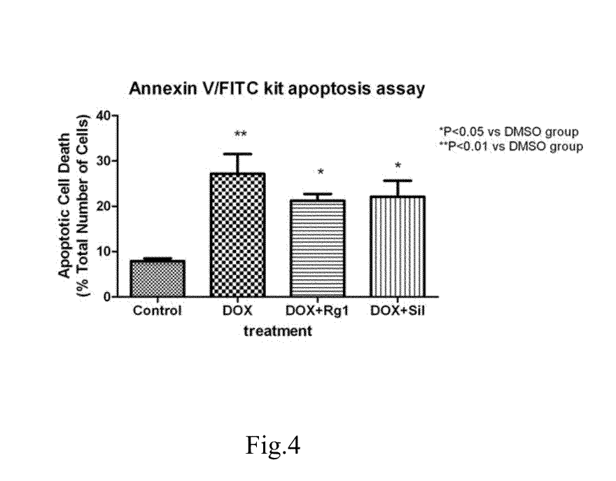

Apoptosis rate of myocardial cells pretreated with Rg1 at a concentration of 200 μM is shown in FIG. 4. The result shows that Rg1 can decrease apoptosis rate of myocardial cells going through hypoxia and reoxygenation.

In short, significant cell apoptosis was observed among myocardial cells induced by 3-hour hypoxia and 3-hour reoxygenation. Rg1 at concentrations of 100 and 200 μM, and Re at concentration of 200 μM were shown to significantly inhibit apoptosis of myocardial cells under hypoxia condition, as compared with the control group.

### 4.2 Study on Cell Cytotoxicity

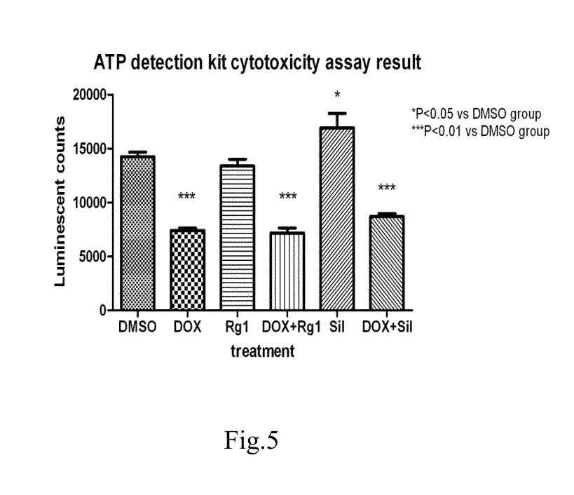

Cytotoxicity of myocardial cells pretreated with Rg1 is shown in FIG. 5. The result shows that Rg1 can decrease the cytotoxicity of myocardial cells going through hypoxia and reoxygenation.

### 4.3 Study on Mitochondrial Membrane Potential Changes

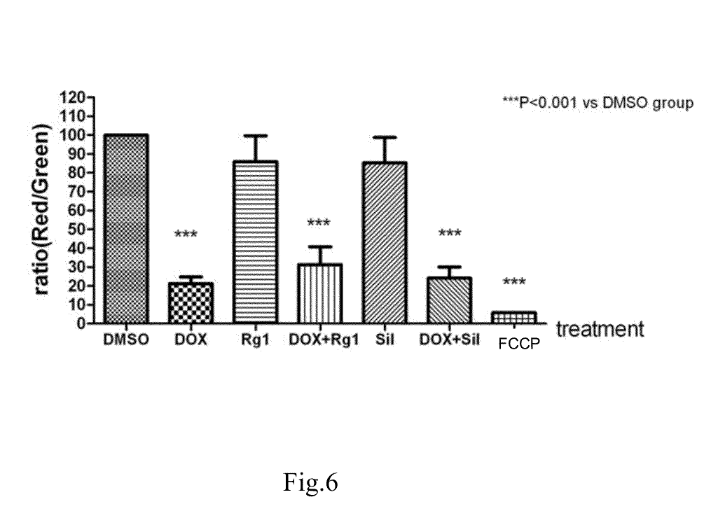

Mitochondrial membrane potential changes by flow cytometer were detected by JC-1 as shown in FIG. 6. The result shows that Rg1 can inhibit the decrease of mitochondrial membrane potential changes in myocardial cells going through hypoxia and reoxygenation.

From this study, Rg1 at concentrations of 100 and 200 μM and Re at concentration of 100 and 200 μM were shown to significantly reduce the decrease of mitochondrial membrane potential changes, as compared with the control group.

### 4.4 Study on ROS Release

Cells were harvested and a single cell suspension was produced by gently pipetting up and down suspension cells or by fully detaching adherent cells. Cells in culture media were stained with 20 μM DCFDA and incubated for 30 minutes at 37° C. After staining, cells were treated with Rg1, DOX or Sil. Cells were gently pipetted to produce single cell suspension. Cells were then analyzed by flow cytometer in which forward and side scatter gates were established to exclude debris and cellular aggregates from the analysis. DCF was excited by the 488 nm laser and detected at 535 nm (typically FL1). 10,000 cells were analyzed per experimental condition. The whole study was repeated 3 times.

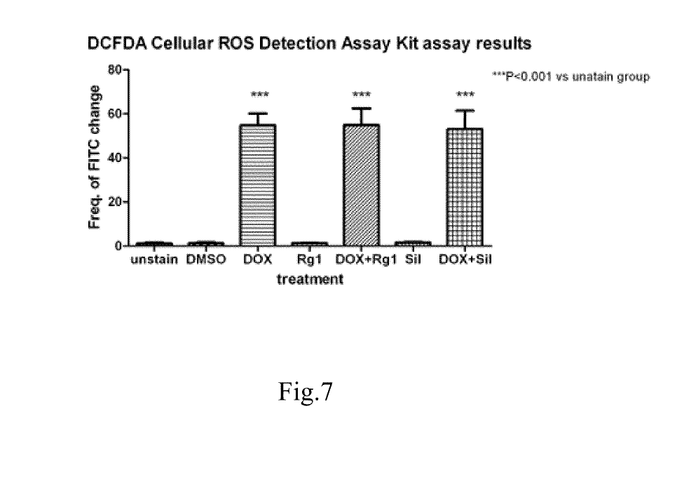

ROS detection was determined as shown in FIG. 7. The result shows that Rg1 can decrease the release of ROS.

From this study, Rg1 at concentrations of 100 and 200 μM and Re at concentration of 100 and 200 μM were shown to significantly reduce the release of ROS, as compared with the control group.

### Example 5

### Analysis for Mark on Mitochondria of Living Cells with Continuous Image Recording

70-80% fused cells (H9C2 cell line) growing in good condition were used. Cells were digested by trypsin and adjusted to a concentration of 5×10⁴/ml, and seeded to 35 mm glass-bottomed petri dishes. Cells were incubated at 37° C. and 5% CO₂ for 24 hours. Cells with good adherent condition as observed by inverted microscope were treated under hypoxia conditions for different time periods. Petri dishes were quickly placed in the cell culture chamber of Delta Vision Personal DV workstation (temperature: 37° C.). Supernatant was removed and medium was replaced with reoxygenation medium containing Mito Tracker Red CMTMRos mitochondria labeled probes (final concentration: 100 nM).

Continuous video recording was carried out on cells loaded with fluorescent probes (including contrast imaging and fluorescence imaging of cells under the same magnification). Five random fields of vision of each petri dish were selected and the data was collected at multiple points, while the petri dishes were instantly recorded during the continuous reoxygenation period. Z section standard 3D image was selected for producing multiple images with different z-axis information. After deconvolution by softWoRx at a later stage, a 2D image was produced to be analyzed by fluorescence intensity software.

The detection of mitochondrial activity of single cells by fluorescence image was described as follows. Based on the comparison of the element calculation of each image with that of the previous and subsequent images, upper and lower displacements are calculated as movement of mitochondria. Element calculation of the previous image and the subsequent image which do not overlap with each other was regarded as a change of mitochondrial movement. Mitochondrial movement curve (MMC) was obtained by continuously calculating elemental displacement difference between each image and the previous one thereof. As mitochondria were moving or swinging, there were some differences or volatility for the same fluorescent focusing plane at different time points. Automatic focusing function was selected for fixing the focusing plane. At the same time the influence of micro-movement on focusing was avoided. Instant fluorescent image was recorded by delayed sequence recording adhered with the software and fluorescent intensity thereof was calculated. Frequency of mitochondrial division and fusion was evaluated as follows. Five fields of vision of the same cell were randomly selected. The occurrences of mitochondrial division and fusion were respectively calculated at continuous time points. The average of the occurrence was divided by the length of time period to obtain the frequency.

Mitochondrial fusion and division of myocardial cells were recorded and the effects of the seven components of protopanaxatriol-type ginsenosides on dynamic change of mitochondria were analyzed. The result shows that Re has the strongest activity of inhibiting the increase of mitochondrial division induced by hypoxia, resulting in the protection effect on ischemic myocardium.

### Example 6

### Method of Fixing Cytoskeleton Microfilament of Myocardial Cell and Fluorescent Staining of Nucleus

Cells were seeded into 6-well plates with coverslips placed thereon and incubated under normal condition for 48 hours. Upon incubation, they were treated under different conditions in which they were respectively fixed by 4% paraformaldehyde. Cells were washed 2-3 times with 50 mM glycine solution in order to neutralize paraformaldehyde solution. Cells were then perforated by PBS solution containing 0.1% Triton X. Cytoskeleton microfilament F-actin was marked by Phalloidin green fluorescent probe while nucleus was marked by DAPI blue fluorescent probe, and staining was carried out at room temperature. Upon washing by PBS twice, the marked components were dried in the air, covered by coverslip, sealed by ProLong® Gold reagent, and then observed under fluorescent microscope and pictures thereof were taken.

### Example 7

### Determination of Expression Levels of Proteins Related to Mitochondrial Division Fusion and Movement, and Apoptosis of Cells by Western Blotting

Myocardial infarction tissues of mice were extracted and washed by precooled PBS, followed by the addition of pre-cooled lysis buffer in a proportion of 100 mg/ml. The tissues were then cut into pieces and the cooled homogenate thereof was centrifuged at 14,000 rpm for 10 minutes. Supernatant was taken as the exacted total myocardial protein.

Protein was quantified and 2× loading buffer was added to the extract containing the same protein quantity, which was then heated at 100° C. for 5 minutes. According to the classical method of Laemmli, 50 μg proteins were added to each loading slot of 10% SDS-PAGE for electrophoresis. After electrophoresis, proteins were transferred onto PVDF membrane by wet methods. Membrane was blocked with TBS solution containing 5% fat-free milk at room temperature for 1 hour, followed by the addition of the corresponding antibody. The antibody of anti-bal-2, anti-bax, anti-caspase-3, anti-caspase-9, anti-PI3K, anti-Akt, anti-Erk, and anti-eNOS were incubated with PVDE membrane at 4° C. overnight. Membrane was washed with TBST buffer solution (0.1% TWEEN 20) three times, 5 minutes for each washing. Membrane and horse radish peroxidase (HRP)-labeled-antibody were co-incubated at room temperature for 2 hours. After the membrane was washed three times, fluorescent measurement was carried out using ECL immune luminescence reagent and developed with film washing. GAPDH was used as internal reference and optical density of protein bands was analyzed by software Quantity One.

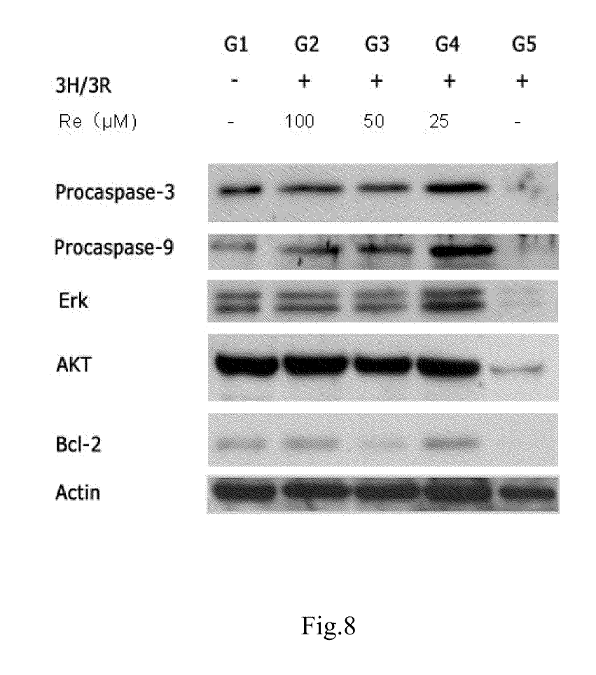

The effects of components of protopanaxatriol-type ginsenosides on the expression level of bal-2, procaspase-3, procaspase-9, Akt, and Erk in myocardial cells treated with 3-hour hypoxia and 3-hour reoxygenation conditions were shown in FIG. 8. The results show that Re, among the seven components, can regulate the mitochondrial-induced pathway of myocardial cells treated with hypoxia and reoxygenation conditions.

The activity of Re is the strongest among the seven components. Re at concentrations of 25, 50 and 100 μM are shown to regulate the expression level of tested protein as shown in FIG. 8. Rg1 at concentration of 100 μM has significant protection for myocardial cells induced by hypoxia and reoxygenation.

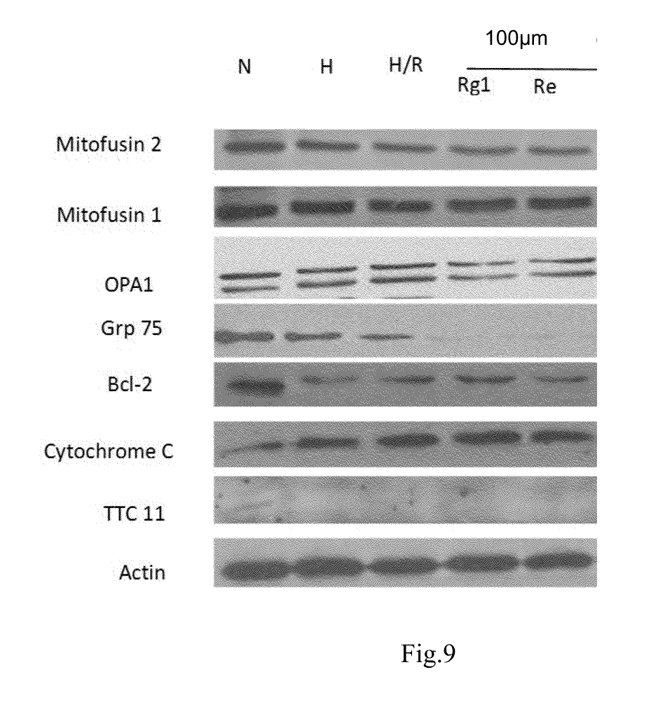

The effects of components of protopanaxatriol-type ginsenosides on the expression level of Fis1, Drp1, Mfn1, OPA1 and Miro1 in the mitochondria of myocardial cells under hypoxia and reoxygenation conditions were analyzed by western blotting, with result as shown in FIG. 9. The results show that Rg1 and Re can exert anti-myocardial-ischemia function via binding with Mitofusin 2 (Mfn2) in mitochondrial.

### Example 8

### Determination of mRNA for Proteins Related to Mitochondrial Division and Fusion by RT-PCR Method

After cells in the logarithmic growth phase were treated for hypoxia and reoxygenation, Trizol was added and the cells were pipetted repeatedly. Cells were then transferred into 1.5 ml centrifuge tubes. Chloroform was added into the tubes and then tubes were placed at room temperature for 5 minutes after rigorous vibration. The tubes were centrifuged at 4° C. and 12,000 rpm for 15 minutes. The upper aqueous phase was extracted and isopropanol was added thereto and well mixed. The mixture was placed still at room temperature and centrifuged. Precipitate was washed twice with 75% ethanol and upon drying, precipitate was dissolved with RNAase-free ultra-pure water. Purity of total RNA was determined by ultraviolet spectrophotometer. cDNA template was synthesized according to the instruction of RevertAid™ First Standard cDNA Synthesis Kit of MBI company. The product was amplified by PCR and then subjected to agarose gel electrophoresis and pictures thereof were taken with EB.

### Example 9

### Statistical Analysis

Experimental data are presented as average±standard deviation, in which N denotes the size of samples. Statistic software SPSS14.0 was used and multiple comparisons among multiple groups were analyzed by variance analysis and post Hoc, Tukey's test. Comparison between two groups was analyzed by t-test where there will be statistical significance for the comparison when P<0.05.

### Example 10

### Purity of components of protopanaxatriol-type ginsenosides

The purities of the seven components, Re, Rf, Rg1, 20(S)-Rg2, 20(R)-Rg2, 20(S)-Rh1 and 20(R)-Rh1 from protopanaxatriol-type ginsenosides were determined by HPLC and the purities of these components were above 97%.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference to describe and disclose specific information for which the reference was cited in connection with.

All references cited above and in the following description are incorporated by reference herein. The practice of the invention is exemplified in the following non-limiting examples. The scope of the invention is defined solely by the appended claims, which are in no way limited by the content or scope of the examples.

1. A method of identifying a therapeutic drug candidate for preventing and/or treating hypoxia-related heart disease comprising:
a) pre-treating myocardial cells with said drug candidate;
b) treating said cells under hypoxia and reoxygenation condition;
c) detecting whether said drug candidate binds with mitofusin-2 in mitochondria; and
d) identifying a drug candidate that performs said binding action of step (c).

2. The method according to claim 1, wherein said step (c) further comprises a step of detecting whether said drug candidate inhibits apoptosis of said cells; wherein a drug candidate is identified as a therapeutic drug for treating said hypoxia-related heart disease if said drug candidate can perform both inhibition of apoptosis and binding action of step (c).

3. The method according to claim 1, wherein said hypoxia-related heart disease is selected from a group consisting of ischemic myocardial disease, myocardial ischemia reperfusion injury and altitude sickness.

4. The method according to claim 1, wherein said myocardial cells are treated under hypoxia condition for 1-3 hours.

5. The method according to claim 4, wherein said myocardial cells are treated under hypoxia condition for 3 hours.

6. The method according to claim 1, wherein said myocardial cells are treated under reoxygenation condition for 1-4 hours.

7. The method according to claim 6, wherein said myocardial cells are treated under reoxygenation condition for 3 hours.

8. The method according to claim 1, wherein said myocardial cells are treated under hypoxia condition for 3 hours and reoxygenation condition for 3 hours.

9. A kit for screening a therapeutic drug candidate for preventing and/or treating hypoxia-related heart disease, comprising myocardial cells pretreated with said drug candidate and treated under hypoxia and reoxygenation conditions; and a protocol for comparing cell viability of said myocardial cells pretreated with said drug candidate and myocardial cells not pre-treated with said drug candidate under hypoxia and reoxygenation conditions, wherein an increase in cell viability of myocardial cells pretreated with said drug candidate over those without pre-treatment is indicative of presence of a prevention/treatment effect of said drug candidate.

10. The kit according to claim 9, wherein said cells are originated from H9C2 or HL-1 cell line.

11. The kit according to claim 9, wherein said hypoxia-related heart disease is selected from a group consisting of ischemic myocardial disease, myocardial ischemia reperfusion injury and altitude sickness.

12. The kit according to claim 9, wherein said cells are treated under hypoxia condition for 1-3 hours.

13. The kit according to claim 12, wherein said cells are treated under hypoxia condition for 3 hours.

14. The kit according to claim 9, wherein said cells are treated under reoxygenation condition for 1-4 hours.

15. The kit according to claim 14, wherein said myocardial cells are treated under reoxygenation condition for 3 hours.

16. The kit according to claim 9, wherein said cells are treated under hypoxia condition for 3 hours and reoxygenation condition for 3 hours.

17. A method of identifying a therapeutic drug candidate for preventing ischemia reperfusion injury in ischemic myocardium comprising:
a) pre-treating myocardial cells with said drug candidate;
b) treating said cells under hypoxia and reoxygenation condition;
c) detecting whether said drug candidate binds with mitofusin-2 in mitochondria; and
d) identifying a drug candidate that performs said binding action of step (c).

18. The method according to claim 17, wherein said step (c) further comprises a step of detecting whether said drug candidate inhibits apoptosis of said cells; wherein a drug candidate is identified as a therapeutic drug for preventing said ischemia reperfusion injury in ischemic myocardium if said drug candidate can perform both inhibition of apoptosis and binding action of step (c).

19. The method according to claim 17, wherein said myocardial cells are treated under hypoxia condition for 1-3 hours.

20. The method according to claim 19, wherein said myocardial cells are treated under hypoxia condition for 3 hours.

21. The method according to claim 17, wherein said myocardial cells are treated under reoxygenation condition for 1-4 hours.

22. The method according to claim 21, wherein said myocardial cells are treated under reoxygenation condition for 3 hours.

23. The method according to claim 17, wherein said myocardial cells are treated under hypoxia condition for 3 hours and reoxygenation condition for 3 hours.
